(19) United States
(12) Patent Application Publication
(10) Pub. No.: US 2011/0245648 A1
(43) Pub. Date: Oct. 6, 2011

(54) Biosensor Remote Collection Packaging System with Bioinformatics Processing

(76) Inventor: Stanford P. Hudson, Dallas, TX (US)

(21) Appl. No.: 12/753,553

(22) Filed: Apr. 2, 2010

(51) Int. Cl. A61B 5/04; H01R 43/00

(52) U.S. Cl. 600/391; 29/825

(57) ABSTRACT

A biosensor kit and method for use thereof for at-home collection of biosensor data having one or more biosensors with a top patch-shaped layer affixed to a bottom patch-shaped layer, the layers being made of cushioning sheet material, such as medical foam tape, and having a biosensor circuit disposed between the layers such that the top layer and bottom layer form a protective, dual-mode encapsulation of the biosensor circuit for cushioning against the skin or a garment of a wearer, and for protection of the biosensor circuit during shipment and handling. The kit further includes a carrier card of suitable size to receive and carry the biosensors in a substantially flat, co-planar arrangement in a substantially flat envelope or pouch for shipment through flat piece mail or postal service. The cushioning sheet material of the biosensor layers is sufficient packing material to avoid requiring additional protective packing material.

### CROSS-REFERENCE TO RELATED APPLICATIONS

None.

### FEDERALLY SPONSORED RESEARCH AND DEVELOPMENT STATEMENT

Not applicable.

### MICROFICHE APPENDIX

Not applicable.

### INCORPORATION BY REFERENCE

None.

### BACKGROUND OF THE INVENTION

1. Field of the Invention

This invention relates to arts and technologies for biosensor packaging systems for bioinformatics processing.

2. Background of the Invention

Many biosensor systems are well known for use in controlled medical environments such as diagnostics laboratories, hospitals, clinics, and physicians' offices. Such biosensor systems may be large and expensive, and relatively immobile. Their costs of acquisition and operation are recouped by the diagnostic lab, hospital, clinic, or physician by having patients come to the facility where they are located for testing. A “sleep lab” is one such facility where many costly and large sensor systems are used to diagnose sleep disorders while a patient slumbers in a bed at the facility. Bioinformatics functionality such as data collection, data processing, and report generation, are typically provided in an integrated fashion with the biosensor system.

Other large and costly biosensor systems include, but are not limited to, systems for monitoring and studying heart functions, brain functions, and pregnancy-related conditions. The technology and usage models of these systems generally involve acquisition of a costly and somewhat large or immobile sensor collection and bioinformatics processing system, location of the system at a commonly-accessible medical facility, and operation and usage of the system by trained medical staff with patients traveling from their home or residence to the location of the medical facility in order to be close enough to the system for sensor application and data collection.

### SUMMARY OF THE INVENTION

A biosensor kit and method for use thereof for at-home collection of biosensor data having one or more biosensors with a top patch-shaped layer affixed to a bottom patch-shaped layer, the layers being made of cushioning sheet material, such as medical foam tape, and having a biosensor circuit disposed between the layers such that the top layer and bottom layer form a protective, dual-mode encapsulation of the biosensor circuit for cushioning against the skin or a garment of a wearer, and for protection of the biosensor circuit during shipment and handling. The kit further includes a carrier card of suitable size to receive and carry the biosensors in a substantially flat, co-planar arrangement in a substantially flat envelope or pouch for shipment through flat piece mail or postal service. The cushioning sheet material of the biosensor layers is sufficient packing material to avoid requiring additional protective packing material.

### BRIEF DESCRIPTION OF THE DRAWINGS

The following detailed description when taken in conjunction with the figures presented herein provide a complete disclosure of the invention.

FIG. 1 shows an overview of the new biosensor and bioinformatics system and the model for usage.

FIG. 2 provides an illustration of a biosensor kit according to at least one embodiment of the present invention.

FIG. 3 shows a top-view of a pair of Restless Leg Syndrome (RLS) biosensors.

FIG. 4 illustrates a method of manufacture of a biosensor in at least one embodiment of the invention.

FIG. 5 illustrates an example patient questionnaire.

FIG. 6 illustrates an example biosensor shipping carrier card.

FIGS. 7, 8 and 9 set forth logical processes according to at least one embodiment of the invention for end-user usage of the biosensor system.

### DESCRIPTION OF THE INVENTION

The inventors of the present invention have recognized that the aforementioned technology and usage models of common biosensor and bioinformatics systems that generally involve acquisition of a costly and somewhat large or immobile sensor collection and bioinformatics processing system, location of the system at a commonly-accessible medical facility, and operation and usage of the system by trained medical staff with patients traveling from their home or residence to the location of the medical facility in order to be close enough to the system for sensor application and data collection, is problematic in that it contributes to several problems in the art:

(a) it is inconvenient for many patients to travel to the location of the system;

(b) it can be expensive for non-ambulatory patients to travel to the location of the system;

(c) it is expensive to train and maintain staff skills for direct operation of complicated and advanced biosensor and bioinformatics systems; and

(d) the foregoing costs and expenses may conspire to tempt a medical professional to over-use such a system (e.g. prescribe use for the system when not clearly medically indicated or justified) in order to recoup the cost of the system and the specially-trained staff.

Having recognized these problems in the art, the inventors set about to develop new biosensor technologies and bioinformatics processing usage models which would:
(a) provide for increased patient convenience by providing patients with a biosensor which is suitable for application and use in a home environment by an untrained person such as the actual patient or a lay caregiver;
(b) through use of the at-home sensor of (a), eliminates the requirement for non-ambulatory patients to travel to the location of the bioinformatics processing system;
(c) reduces the cost of bioinformatics processing and sensor application by eliminating the need for physicians, clinics, labs, and hospitals to maintain specially trained staff through providing bioinformatics processing in a service model, preferably remote to the actual prescribing physicians' offices, clinics, labs, and hospitals;
(d) thereby removing temptations to a medical professional to over-use such a system due to elimination of large outlays to acquire a biosensor and bioinformatics system, and elimination of keeping specially-trained personnel on staff.

These advantages, and others, will be apparent in the following paragraphs. In the exemplary embodiments, the inventors provide methods, materials, and processes suitable for use in the sleep disorder study, diagnosis and treatment medical fields, especially Restless Legs Syndrome (RLS). However, the invention may be equally beneficial to other fields of medicine, such as cardiology, neurology, obstetrics, as well as many other fields which benefit from the use of biosensors and bioinformatics.

### Overview of the Biosensor and Bioinformatics System and Usage Models

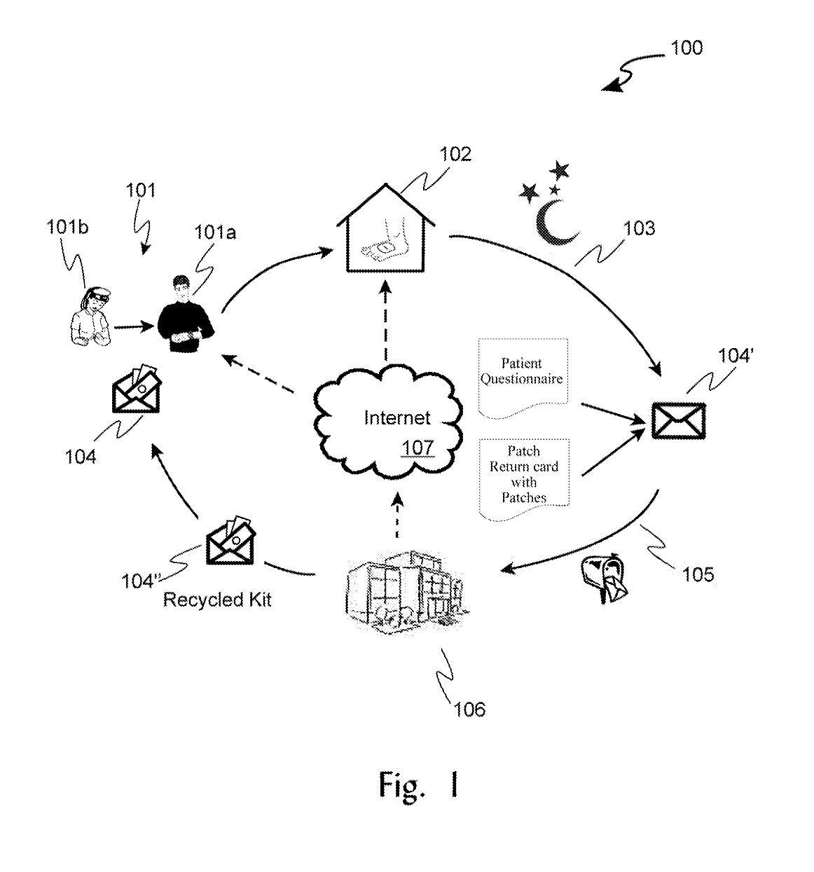

Turning to FIG. 1, an overview of the new biosensor and bioinformatics system and the model for usage (100) is shown. When a patient (101a) visits a medical facility (101) such as a physician's office, laboratory, clinic, or hospital, for initiation of biosensor-based testing and diagnosis, he or she is provided a fresh “take home” biosensor kit (104) by a staff member (101b) at the medical facility. Because of the design of the biosensor kit to be applied and used by non-medical personnel, such as by the patient himself or herself, the staff member (101b) need not be specially trained in application and use of the biosensor, nor specially trained in the operation of the bioinformatics processing system associated with the particular test or diagnostic procedure. In our example embodiment, the patient is given a take-home kit containing a number of components, including, but not limited, to one or more self-adhesive limb movement biosensor “patches” which will be described in greater detail in the following paragraphs.

The patient then returns to his or her home or residence (102), whereby the patient receives instructions on the use and application of the sensor(s), and may be required to complete certain questions on a questionnaire, such as time of application and activation of the biosensor and “vital conditions” of the patient at such time (temperature, diet, pain levels, etc.).

Next, the patient activates and applies the biosensor for a period of time or throughout an activity (103), such as overnight while sleeping in the example embodiment of an RLS biosensor.

At the conclusion of the activity or sensor operation period (103), the patient then removes the biosensor(s), applies them to a biosensor carrier card to defeat an adhesive surface, and preferably completes the rest of the questionnaire, such as recording time of biosensor removal, and patient “vitals”. These are then placed in a “return” package (104′) included in the original fresh kit (104), and shipped via postal or courier service (105) to a bioinformatics data processing center (106). The “return” package is preferably self-addressed to the bioinformatics data processing center, and is provided with pre-paid, collect-on-delivery, or addressee-charged postage or courier charges. While the term “return” is used here because, from the patient's perspective, it may appear that he or she is returning the sensors to the lab or medical facility, but in reality, he or she is forwarding the used sensors and information to the bioinformatics processing center.

Upon receipt of the used kit (104′), the bioinformatics data processing center removes the questionnaire and biosensors from the return card and uploads or reads the biosensor data from the units into a computer equipped with suitable bioinformatics programs. In the exemplary embodiment, the bioinformatics data processing center is equipped with suitable computers and programs to analyze limb movement sensor data and to produce one or more graphic reports for assisting a physician in the diagnosis and treatment of RLS.

At this point, depending on the cost of the sensors and the materials employed in the sensors, the biosensor units may be discarded, or they may be erased and “cleared” to a “fresh” state, placed in a recycled “fresh” kit (104″), and shipped to a medical facility where the entire cycle (100) may be repeated.

It is important to note that this process (100) is enhanced for cost and usage by one or more features of the invention, described in more detail in the following paragraphs, which may be embodied together or in sub-combinations:
(a) medical facility cost is reduced by providing a biosensor which is easy enough to use by a patient without instructions beyond a printed instruction sheet or included video media (DVD, Thumbdrive, etc.);
(b) shipping costs are reduced by providing a biosensor having a weight and thickness which allows for mailing or shipping in a flat envelope without a special surcharge;
(c) shipping costs are further reduced by producing a biosensor using materials which, in a first manner of usage, act as a comfortable, hygienic, hypo-allergenic, pliable self-adhesive patch, and which, in a second manner of usage, act as sufficient impact absorption to protect sensitive electronics such as accelerometers, batteries, electrical pickups, temperature sensors, and strain gauges, during normal shipping and handling, thereby reducing or eliminating the need for supplemental impact packaging such as foam, air bags, or bubble wrap; and
(d) bioinformatics processing is handled remotely from the medical facility, providing reports via electronic communications such as the Internet, email, etc., thereby reducing the equipment cost, size, and training expenses of having the bioinformatics co-located with the biosensors at the medical facility.

These process improvements and efficiencies are directly enabled and provided by certain structural and operational aspects of the invention's several embodiment aspects which will be described in full detail in subsequent paragraphs.

### Biosensor Kit

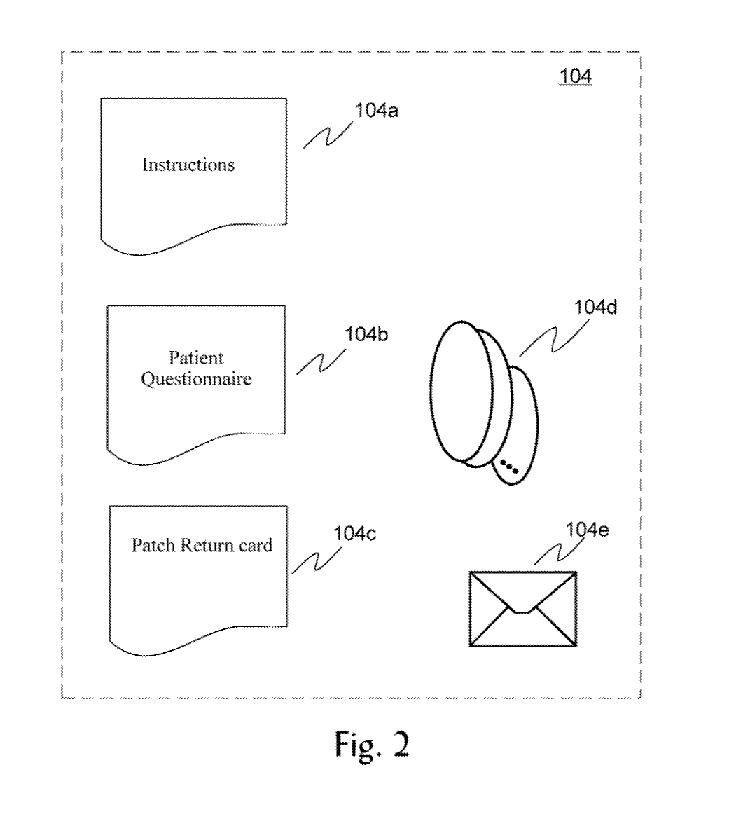

FIG. 2 provides an illustration of a biosensor kit (104) according to at least one embodiment of the present invention. This kit is exemplary only, as the actual contents of other kits for other types of medical studies may include more or less components without departing from the spirit and scope of the present invention.

The biosensor kit preferably includes instructions (104a) for the non-medically-trained user, such as a patient or lay caregiver, a questionnaire or data collection form (104b), one or more biosensors (104d), and a return card (104c) for acting as a carrier of the self-adhesive biosensors. Also included in the exemplary embodiment is a postage-paid pre-addressed envelope (104e) for sending the used biosensors and completed questionnaire to the bioinformatics processing center.

### Exemplary Biosensor

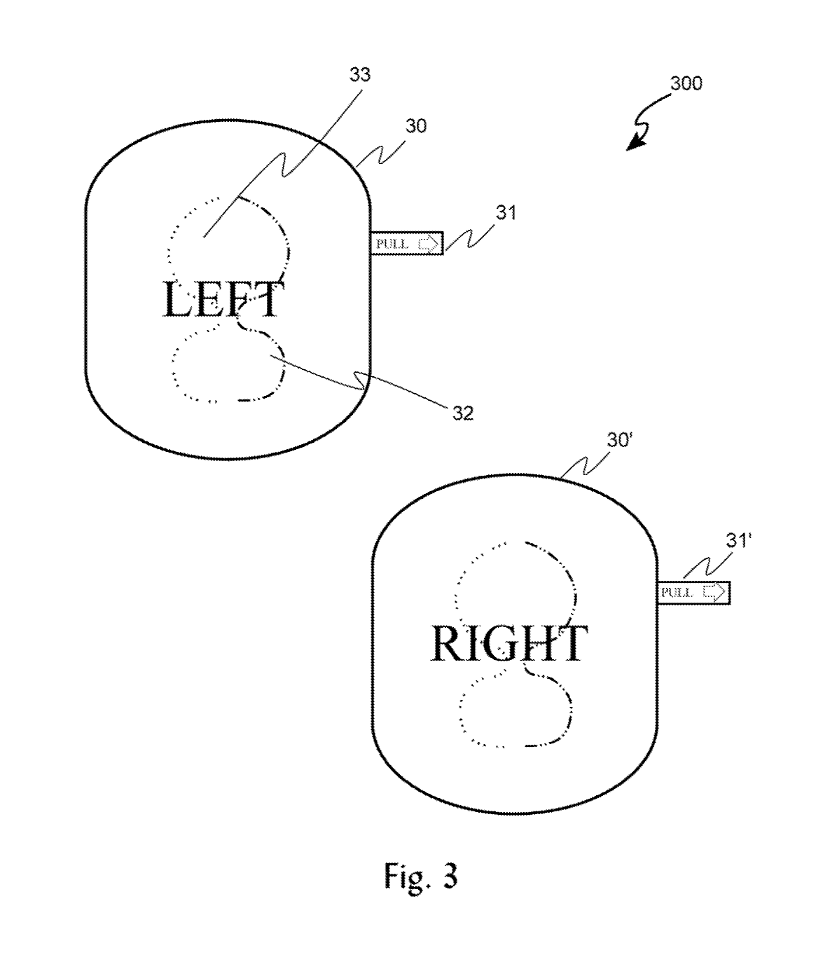

As already stated, an exemplary embodiment is a limb movement sensor system for the measurement, diagnosis, and treatment of sleep disorders such as Periodic Limb Movement Disorder (PLMD) and Restless Leg Syndrome (RLS). FIG. 3 shows a top-view of a pair of RLS biosensors (300).

In this embodiment, each “patch” (30, 30′) has a battery isolation strip (31, 31′) which prevents an internal battery from completing a circuit to an internal biosensor circuit utilizing an accelerometer to measure and collect limb movement data. The battery and circuit are fully enclosed (32) in the patches in a package which may be referred to as a “blister” pack, described in more detail in the following paragraphs.

To activate each sensor, the user must only pull the battery isolation strip out of the patch, thereby completing a conductive path from the battery to the biosensor circuit. Alternatively, for extremely low power circuits or short-duration studies, the battery may be engaged continuously. Or, a switch may be employed in place of the battery isolation strips.

To apply the biosensors, a release paper is removed from the bottom surface (not shown), exposing a self-adhesive surface for adhering the patch to a patient's foot, leg, forearm, or hand, for example.

### Method of Manufacture of Biosensor

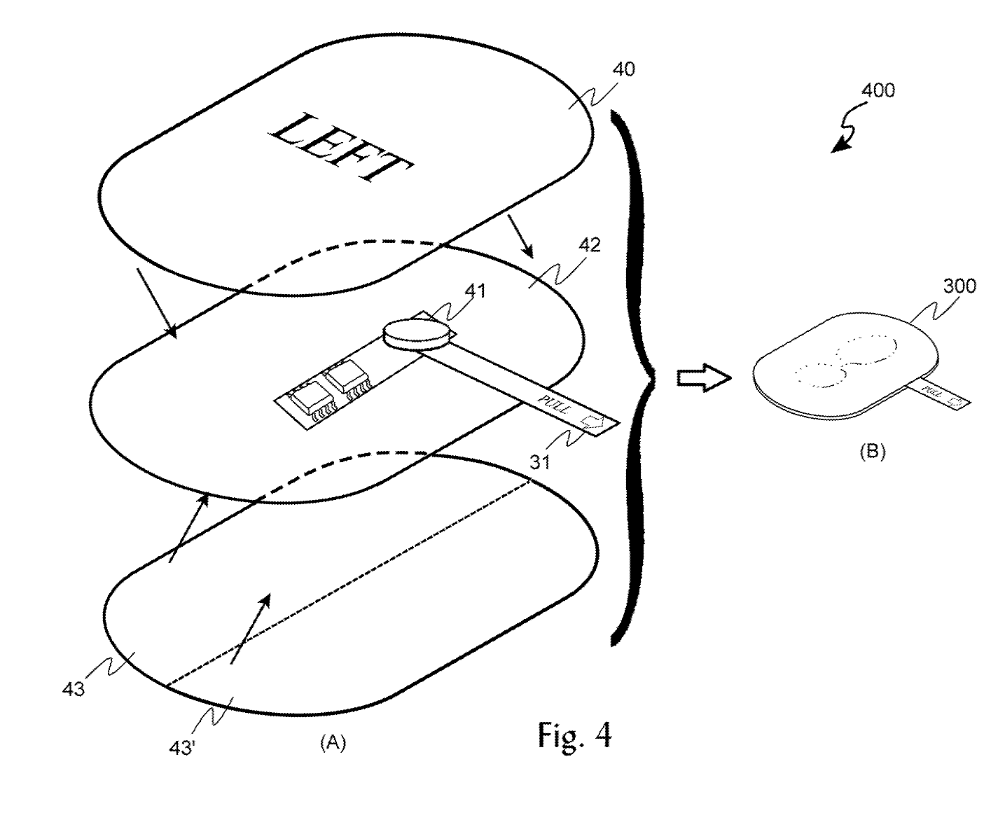

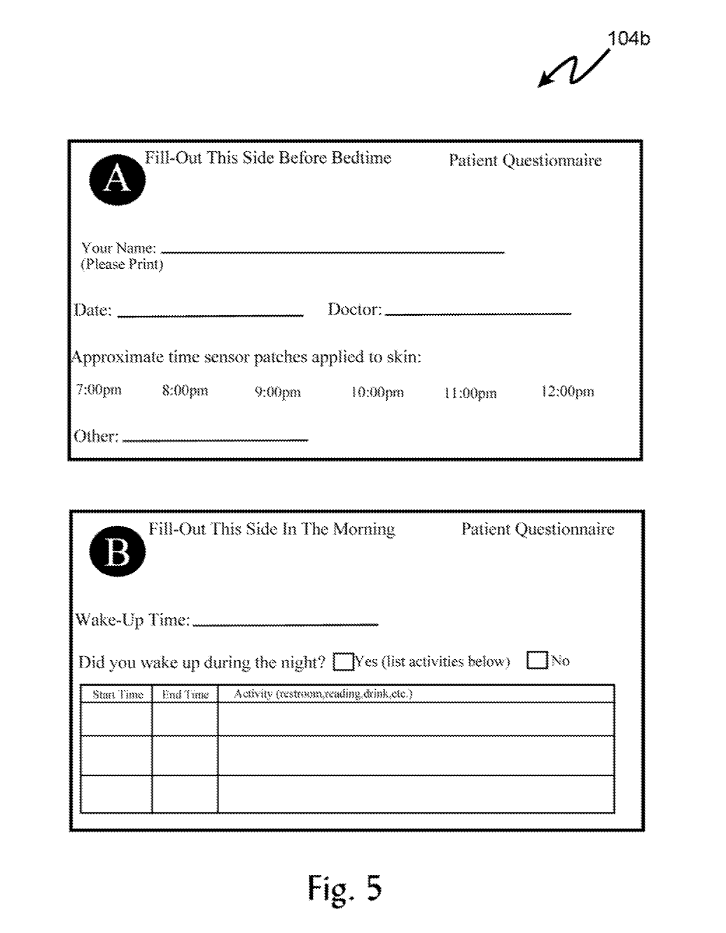

According to another aspect of at least one embodiment of the present invention, a biosensor is manufactured by cutting or forming two patch-shaped pieces (40, 42) of self-adhesive medical foam tape, such as 3M™ No. 9777-L PVC surgical foam tape or similar hypo-allergenic, hygienic foam tape, as illustrated in FIG. 4a. Alternatively, medical-grade cushioning sheet material may be used as well, such as cast padding or fibrous foot corn patch material, and suitable adhesive may be used as appropriate to affix the layers together and to the patient.

A biosensor circuit board with battery (41) is preferably disposed between the top patch layer (40) and the bottom patch layer (42) by adhering the top layer to the bottom layer, thereby by capturing the circuit board “sandwiched” between the top and bottom layers but sealed around the circuit board, thus forming a padded blister packaging for the circuit board. In the exemplary embodiment, a battery isolation strip (31) extends from the edge of the pair of layers of the patch in order to allow the user to pull the strip out to activate the biosensor circuit board. Also according to this exemplary embodiment, the biosensor board may be constructed using a flexible printed circuit board (“flex circuit”) to enhance comfort of the patch when worn by a user, and to promote conformance of the patch to non-planar surfaces of a patient's body, such as the top of the arch of a foot.

In this exemplary embodiment, release paper (43, 43′) is provided on the bottom surface of the bottom layer (42), which, when removed by the lay caregiver or the patient, exposes an adhesive for affixing the patch to a body part, such as the top of a foot.

The assembled patch (300) as shown in FIG. 4b resembles a thick, flexible, impact-absorbing patch with self-adhesive release paper on the bottom side, with a battery activation strip extending from one edge, and potentially with lump or blister in the center (33 in FIG. 3) of the patch. The foam or cushioning sheet material used in the construction of the patch layers acts as a carrier to hold the circuit to the patient's body part, but also serves a second function to shield the sensitive biosensor circuit from impacts during mailing or shipping of the kit to the bioinformatics processing center. By integrating the packing material for the biosensor into the construct of the biosensor itself, costs are reduced by eliminating need for additional packing material in the return envelope, such as bubble wrap, and by reducing the thickness of the return envelope which leads to lower postage costs.

### Patient Data Collection

In many manners of usage of biosensors according to the present invention, some information may be needed from the patient or lay caregiver about the application and activation of the biosensor, and potentially vital statistics about the patient, such as date and time of activation, patient's name, and details of nighttime activities, as in the example embodiment for RLS studies, shown in FIG. 104b. A portion of the questionnaire may be completed prior to or at the time of activation and application of the biosensor, and a portion of the questionnaire may be completed upon removal of the biosensor, such as a before bedtime and upon waking, respectively, in the example RLS embodiment.

### Biosensor Return Carrier Card

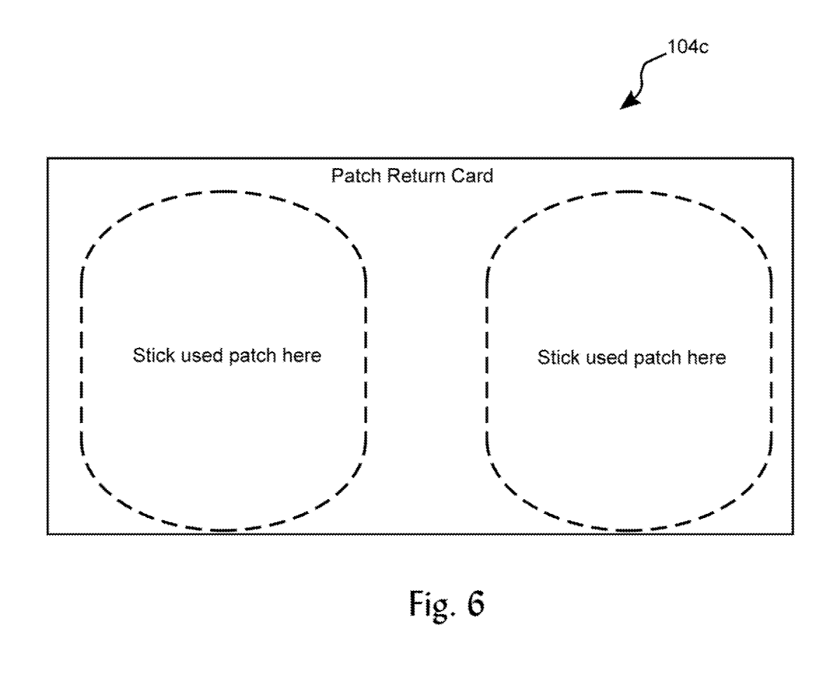

According to another aspect of one embodiment, the “return” package includes a biosensor return carrier card (104c), as shown in FIG. 6, upon which the user may affix the removed and used biosensors using the same self-adhesive bottom surface that was previously used to affix the biosensors to the body part. This card serves several functions in this embodiment, including:
(a) it maintains the relatively flat biosensor patches in a co-planar arrangement for flat packaging into the pre-addressed envelop in order to minimize thickness and postage of the filled envelop;
(b) it organizes the contents of the return package to allow quick and efficient inbound handling at the bioinformatics processing center; and
(c) it occupies the sticky self-adhesive surfaces of the patches after they were removed from the body part in order to keep them from sticking to the inside of the envelop or to each other.

### Biosensor Logical Processes

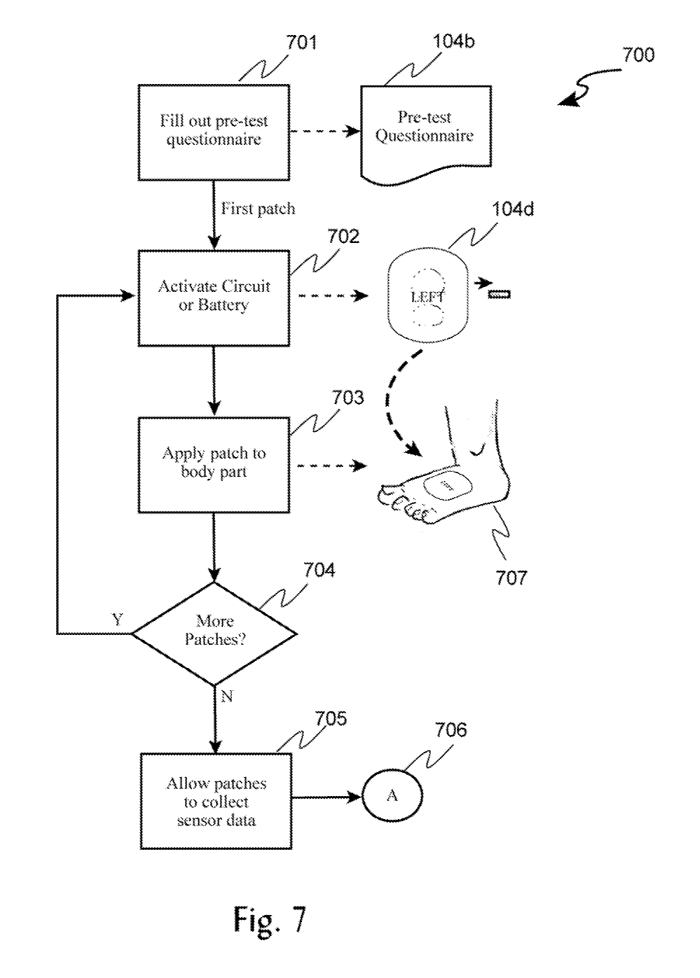

Turning to FIG. 7, a logical process (700) for a manner of usage of at least one embodiment according to the invention is shown. In the example RLS embodiment, the patient completes a bedtime portion of the questionnaire (701), and then activates a first biosensor patch (702) by, for example, removing the battery isolation strip, or alternatively pressing a switch or button. The first biosensor patch is then applied to a body part (703), such as by removing a release paper to expose the bottom adhesive layer and affixing the first patch to a first body part, such as the top of a first foot. If more patches are to be employed (704), then the activation (702) and application (703) steps are repeated until all patches have been activated and applied.

Then, the patient performs an activity for a period of time (705), such as sleeping normally in his or her own bed overnight (rather than in a hospital bed in a sleep lab) in the example embodiment of an RLS system.

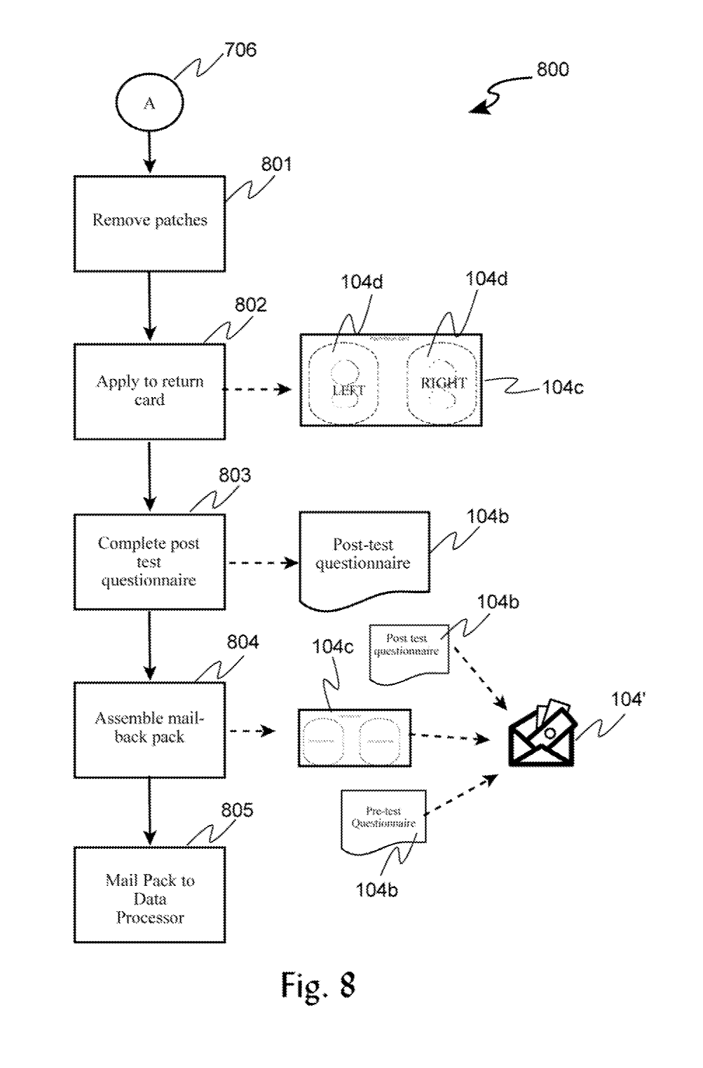

Following completion of the testing or monitoring activity and/or period, the logical process continues (706) as shown (800) in FIG. 8. The user or lay caregiver removes the biosensor patches (801), affixes them to the biosensor carrier card (802), and optionally completes a post-test portion of a patient questionnaire, such as time of patch removal or time of waking. Then, the end-user assembles (804) the mail-back “return” pack as set forth in the user instructions, placing (805) them in the postal service or other courier service for delivery to the bioinformatics processing center, as illustrated more generally in FIG. 1.

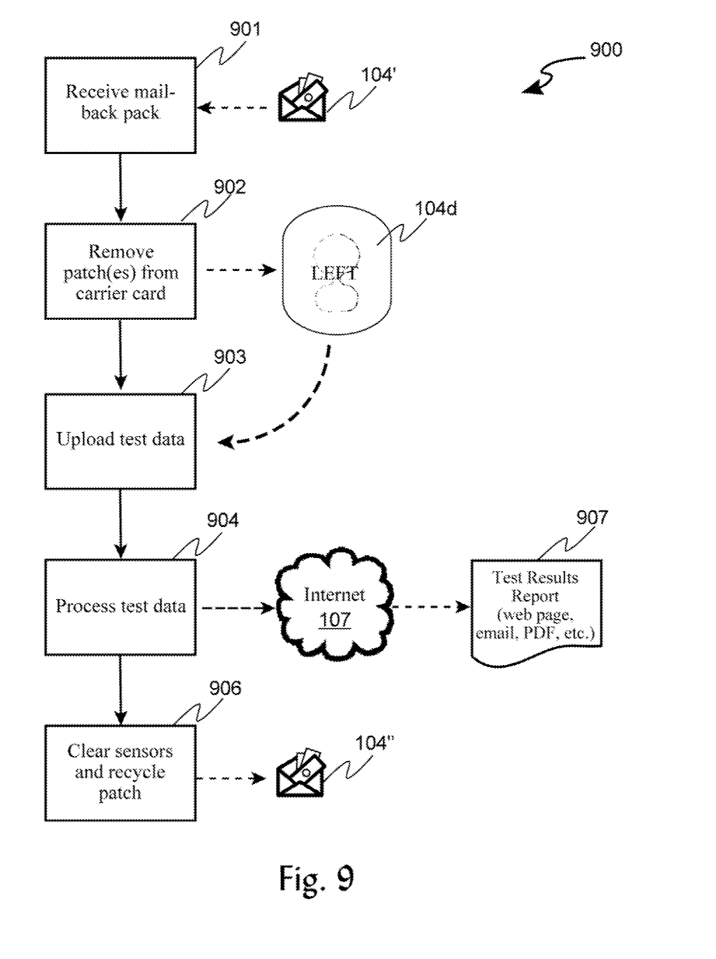

Turning to FIG. 9, a logical process (900) according to at least one embodiment of the invention is shown for bioinformatics data processing and manner of usage of the used biosensors. The mail-back pack (104′) is received (901) by the bioinformatics processing center, and the patches are removed (902) from the pack. Test data is then uploaded (903) from each patch, such as by short range wireless communications such as BlueTooth™ or Radio Frequency Identification (RFID), or by a wired connection such as Universal Serial Bus (USB). The uploaded data is then processed (904) by a computer and one or more computer programs at the bioinformatics processing center (106), and results and reports (907) from which are then preferably made available via an electronic storage medium such as a secure web page on a web server connected to the Internet (107), by secure email, by secure email attachment, or similar, to the physician or medical facility (101), and optionally to the patient at home (102). The reports and results are then available remotely and conveniently to the physician's and/or their staff, and optionally to the patient or the patient's lay caregiver.

According to this example embodiment, the sensors are then “recycled” (906) by clearing their memories, replacing the battery isolation strip, and optionally replacing the battery, in preparation for sending them to another medical facility for re-use. In the example embodiment in which medical surgical foam tape is used for the layers of the biosensor patch, many of these foam tapes are produced on a spool or roll such that their adhesive does not adhere permanently to the top side of the foam. This non-permanent adhesion “to itself” allows for the tape to be removed from the roll, of course. In our embodiment, it also allows the two layers of the patch assembly to be easily separated, revealing the biosensor circuit board and the battery. When the circuit board is exposed, the battery may be serviced by installing a battery isolation strip, and optionally by replacing the battery. Then, the circuit can be readily assembled into fresh patch layers of medical foam tape.

### RLS Biosensor Example Embodiment Details

In the foregoing example embodiments, RLS sensors were described to illustrate at least one manner of usage of the invention. The following paragraphs provide additional details of an actual prototype system produced and tested by the inventors. This prototype was used to prove and perfect the concept, and in no manner represents the limits of the scope of the present invention.

General Description. The periodic leg movement sensor device is designed to sense and log periodic leg movements while the user is sleeping. The sensor device is enclosed in a patch that is attached to the user's skin via adhesive. Alternatively, the patch may be affixed to a conforming garment worn by the user, such as a sock or bandage. After use, data is downloaded from the sensor device to a computer for post-processing and analysis. The typical system is comprised of two (2) sensor devices (patches) that are worn on the left and right foot of the user.

System Components. The periodic leg movement sensor device circuit is comprised of a microprocessor, a tri-axis accelerometer, a non-volatile memory, a battery, and an activation switch. An analog-to-digital (ADC) peripheral internal to the microprocessor is used to sample the outputs from the accelerometer. Preferably, all three axis outputs from the accelerometer are used (X, Y and Z). However, in some embodiments, fewer axes may be suitable and sufficient depending on the application.

The sensor activation switch is, according to one embodiment, provided in the form of a non-conductive battery isolation strip is inserted between a metal plate and a conductor such as a printed circuit board pad or a solder blob to break the connection between a terminal of the battery and the rest of the biosensor circuit. While the connection is broken, no battery power is applied to the circuit.

System Activation. To activate the biosensor, the user removes the non-conductive battery isolation strip when the sensor is ready to be used. Removal of the isolation strip allows the metal plate to conductively contact the PCB pad or solder blob, thereby completing the circuit from the battery to the biosensor circuit, allowing current to flow to the biosensor circuit from the battery, thus powering (activating) the biosensor. The user then applies the biosensor (enclosed in the patch) to the top of the foot.

Data Collection. After activation, the biosensor begins collecting data by sampling outputs by a microprocessor from an integrated accelerometer (via the ADC), for example, at 8 samples per second in one embodiment. Also according to this particular embodiment, there is no filtering or averaging of data. All three outputs are summed together for each sample by the microprocessor, and the absolute difference between the previous summation and the current summation is then compared with a threshold. If exceeded, then it is considered movement. The memory contains 512 bytes. Each bit represents 10 seconds for a total recording time of over 11 hours. If movement occurred within a particular 10-second period, then the bit is set for that period. When a movement occurs, the system will not record any further movements until no movement has occurred for at least 5 seconds. Alternate embodiments may use different filtering, averaging, summation, and recording schemes.

The biosensor continues, according to one embodiment, to collect data and store data until either (1) the non-volatile memory is full, or (2) the system is powered off.

Data Download. Data is downloaded from the biosensor to a computer, such as a Personal Computer (PC) via a serial connection. The biosensor outputs only the contiguous non-FF non-volatile memory values starting at address zero in one embodiment, in which the output is in two-digit hexadecimal format. HyperTerminal™ is used to download the HEX data to a text file, for example.

Data Processing and Analysis. A software program running on the PC extracts the data from the downloaded text file and, using a number of developed algorithms, produces a text file containing:
1. the decimal version of the hexadecimal data;
2. valid leg movements (LMs);
3. valid periodic leg movements (PLMs); and
4. statistics including, but not limited in all embodiments to, total sleep time, LM index (e.g. average number of leg movements per hour of sleep), PLM index (e.g. number of periodic leg movements per hour of sleep), and number of LMs, number of PLMs.

According to our exemplary embodiment, a valid leg movement is determined to be a movement that lasts between 0.5 seconds and 5 seconds. And, a valid PLM sequence is comprised of four or more consecutive valid leg movements which are 5 to 90 seconds apart.

Optionally, one or more histograms may be graphically produced representing any or all of this information, as well, such as by using spreadsheet is used to plot the various information.

### Other Uses

It should be noted that the example embodiments and manners of usage do not define the full scope of the present invention, but provide an illustration of how the invention may be applied to a Restless Leg Syndrome medical art. Other medical arts, including, but not limited to, the following examples may benefit as well:
(a) cardiology studies may be remotely and conveniently conducted on patients by providing a heart monitor version of the biosensor which can be used on the chest, wrist, or neck, such as during normal daily activities;
(b) other forms of sleep disorders, such as sleep apnea may be studied by providing a sound monitor with or without a movement monitor;
(c) nocturnal hypoglycemia may be studied in diabetics by studying shifts in limb movement patterns;
(d) body position may be sensed and recorded during sleep or another activity; and
(e) neurological studies on stroke and seizure-prone patients can be performed using variations of the biosensor patch, including diseases such as Parkinson's Disease and Tourettes Syndrome.

For these reasons, the full scope and spirit of the present invention should be ascertained from the following claims.

1. A biosensor kit for collection of biosensor data comprising:
one or more biosensors comprising at least a top patch-shaped layer affixed to a bottom patch-shaped layer, the layers comprising cushioning sheet material, and having a biosensor circuit disposed therebetween wherein the top layer and bottom layer form a protective, dual-mode encapsulation of the biosensor circuit for cushioning against the skin or a garment of a wearer, and for protection of the biosensor circuit during shipment and handling;
a carrier card of suitable size to receive and carry the one or more biosensors in a substantially flat, co-planar arrangement, by affixing the biosensors to the carrier card; and
an envelope or pouch of substantially flat configuration and dimensions wherein the carrier card with the affixed biosensors are received into the envelope or pouch, and having a suitable seal to contain the carrier card for shipment through flat piece mail or postal service, wherein the cushioning sheet material of the biosensor layers is sufficient packing material to avoid requiring additional protective packing material.

2. The biosensor kit as set forth in claim 1 wherein the cushioning sheet material comprises adhesive medical foam tape.

3. The biosensor kit as set forth in claim 1 wherein the cushioning sheet material comprises adhesive fibrous tape.

4. The biosensor kit as set forth in claim 1 wherein the envelope or pouch comprises a pre-addressed return envelope.

5. The biosensor kit as set forth in claim 1 wherein the envelope or pouch comprises a postage-paid return envelope.

6. The biosensor kit as set forth in claim 1 wherein the bottom layer sheet material further comprises a bottom surface having a non-permanent adhesive initially covered by one or more pieces of release paper, and wherein, following removal of the release paper, the adhesive affixes a biosensor to a wearer, and serves to affix the biosensor to the carrier card.

7. The biosensor kit as set forth in claim 1 wherein the biosensor further comprises a biosensor activation means.

8. The biosensor kit as set forth in claim 7 wherein the activation means comprises a removable battery isolation strip.

9. The biosensor kit as set forth in claim 7 wherein the activation means comprises an encapsulated switch.

10. The biosensor kit as set forth in claim 7 wherein the biosensor circuit comprises a limb movement sensor.

11. A method for remote collection of biosensor data comprising:
providing a biosensor kit to a non-expert user comprising:
one or more biosensors comprising at least a top patch-shaped layer affixed to a bottom patch-shaped layer, the layers comprising cushioning sheet material, and having a biosensor circuit disposed therebetween wherein the top layer and bottom layer form a protective, dual-mode encapsulation of the biosensor circuit for cushioning against the skin or a garment of a wearer, for protection of the biosensor circuit during shipment and handling;
a carrier card of suitable size to receive and carry the one or more biosensors in a substantially flat, co-planar arrangement, by affixing the biosensors to the carrier card; and
an envelope or pouch of substantially flat configuration and dimensions wherein the carrier card with the affixed biosensors are received into the envelope or pouch, and having a suitable seal to contain the carrier card for shipment through flat piece mail or postal service, wherein the cushioning sheet material of the biosensor layers is sufficient packing material to avoid requiring additional protective packing material;
affixing by a non-expert user the biosensor to a portion of the user's body or a user's conforming garment;
collecting and storing biodata for a period of time during a specified activity by the biosensor;
removing by the non-expert user the biosensor from the user's body;
affixing the biosensor to the carrier card;
enclosing and sealing the carrier card in the envelope or pouch;
posting or shipping the envelope or pouch to a data processing center;
processing the biodata by the data processing center to produce one or more reports; and
providing the one or more reports to one or more users.

12. The method as set forth in claim 11 wherein the biosensor comprises a limb movement biosensor patch.

13. The method as set forth in claim 11 wherein the non-expert user comprises a medical patient.

14. The method as set forth in claim 11 further comprising:
erasing or otherwise clearing and resetting the biosensor circuit and biodata;
packaging the biosensor with a fresh carrier card and envelope or pouch to produce a recycled biosensor kit; and
providing the recycled biosensor pouch to an expert user.

15. The method as set forth in claim 14 wherein the expert user is a medically-trained user.

16. The method as set forth in claim 11 wherein the biosensor kit is provided to a non-expert user by an expert user, and wherein affixing the biosensor to the body portion and to the carrier card is provided at non-medical, residential building or facility.

17. A method of manufacturing a biosensor kit for collection of biosensor data comprising:
producing one or more biosensors comprising:
disposing a biosensor circuit on between a top patch-shaped layer and a bottom patch-shaped layer, the layers comprising cushioning sheet material; and
affixing the top patch-shaped layer to the bottom patch-shaped layer forming a protective, dual-mode encapsulation of the biosensor circuit for cushioning against skin or a garment of a wearer, and for protection of the biosensor circuit during shipment and handling; and
providing a biosensor kit comprising packaging the encapsulated biosensors with a carrier card of suitable size to receive and carry the one or more biosensors in a substantially flat, co-planar arrangement, by affixing the biosensors to the carrier card, and with an envelope or pouch of substantially flat configuration and dimensions;
wherein the carrier card with the affixed biosensors are receivable into the envelope or pouch, the envelop or pouch having a suitable seal to contain the carrier card for shipment through flat piece mail or postal service, wherein the cushioning sheet material of the biosensor layers provides sufficient packing material to avoid requiring additional protective packing material.

18. The method as set forth in claim 17 wherein the cushioning sheet material comprises adhesive medical foam tape.

19. The method as set forth in claim 17 wherein the cushioning sheet material comprises adhesive fibrous tape.

20. The method as set forth in claim 17 wherein the envelope or pouch comprises a pre-addressed return envelope.

21. The method as set forth in claim 17 wherein the envelope or pouch comprises a postage-paid return envelope.

22. The method as set forth in claim 17 wherein the bottom layer sheet material is provided with a bottom surface having a non-permanent adhesive initially covered by one or more pieces of release paper, and wherein, following removal of the release paper, the adhesive is suitable to affix a biosensor to a wearer, and serves to affix the biosensor to the carrier card.

23. The method as set forth in claim 17 wherein producing the biosensor comprises providing a biosensor activation means.

24. The method as set forth in claim 23 wherein the activation means comprises a removable battery isolation strip.

25. The method as set forth in claim 23 wherein the activation means comprises an encapsulated switch.

26. The method as set forth in claim 17 wherein producing the biosensor comprises producing a limb movement sensor.
